(19) United States
(12) Patent Application Publication
(10) Pub. No.: US 2021/0343378 A1
(43) Pub. Date: Nov. 4, 2021

(54) MEDICAL STATUS PERSONAL DEVICE

(71) Applicant: AXION PARTNERS LLC, Arlington, VA (US)

(72) Inventors: Jonathan DOYLE, Arlington, VA (US); Damon JACKMAN, Silver Spring, MD (US)

(73) Assignee: AXION PARTNERS LLC, Arlington, VA (US)

(21) Appl. No.: 17/306,088

(22) Filed: May 3, 2021

Related U.S. Application Data
(60) Provisional application No. 63/018,831, filed on May 1, 2020.

(51) Int. Cl. G16H 10/40; G16H 10/65; G16H 40/63; G16H 15/00; G06F 21/32; G06F 1/16; G06K 9/00

(52) U.S. Cl. CPC G16H 10/40; G16H 10/65; G16H 40/63; G06K 9/00013; G06F 21/32; G06F 1/1632; G16H 15/00

(57) ABSTRACT

A personal medical status device attaches to a medical testing kit to receive a test result and status for a medical condition of a user. The personal medical status device authenticates the user to provide the status of the condition using a fingerprint scanner as well as other methods. A clock display provides time related information based on the latest test result. The clock display can show the time from the previous test or time until the next test. A test result display provides a visual indicator of the status of the user without compromising privacy. A proprietary dock allows connection to the medical testing kit while being tamper-proof.

### FIELD OF THE DISCLOSURE

The present disclosure relates to a personal device used to indicate a status of a user with regard to a medical status or condition.

### SUMMARY

A personal medical status device is disclosed. The personal medical status device includes a processor and a memory connected to the processor. The memory stores instructions executable by the processor to receive and process a test results received from a medical testing kit. The memory stores the test result provided by the medical testing kit and a status associated with the test result. The personal medical status device also includes a fingerprint scanner to scan a fingerprint of a user and to authenticate the user with a fingerprint file stored in the memory. The personal medical status device also includes a clock display to indicate a time related to the test result provided by the medical testing kit. The personal medical status device also includes a test result visual display to indicate the status of the test result for the user. The personal medical status device also includes a proprietary dock to attach to the medical testing kit. The proprietary dock receives the test result from the medical testing kit and certifies the test result. The proprietary docket includes a tamper proof feature and only allows data to transfer in one direction from the medical testing kit.

A medical testing and status system is disclosed. The medical testing and status system includes a medical testing kit to determine a status for a medical condition of a user. The medical testing and status system also includes a personal medical status device connected to the medical testing kit.

A method is disclosed. The method includes performing a medical test for a condition of a patient using a medical testing kit. The method also includes connecting a personal medical status device to the medical testing kit. The method also includes receiving a test result for the medical test at the personal medical status device using a proprietary dock. The test result is certified. The method also includes storing the test result on the personal medical status device. The method also includes displaying a time based on the receipt of the rest result with a clock on the personal medical status device. The method also includes indicating a status of the test result using a test result visual display on the personal medical status device. The method also includes authenticating the status of the patient using a fingerprint scanner on the personal medical status device. The status is displayed after verification by the fingerprint scanner.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

Various other features and attendant advantages of the present invention will be more fully appreciated when considered in conjunction with the accompanying drawings.

FIG. 1 illustrates a block diagram of the components of the personal medical status device according to the disclosed embodiments.

FIG. 2 illustrates a personal medical status device according to the disclosed embodiments.

FIG. 3 illustrates a medical testing and status system according to the disclosed embodiments.

FIG. 4 illustrates a personal medical status device attached to a mini-test kit according to the disclosed embodiments.

FIG. 5 illustrates a flowchart for using the medical testing and status system according to the disclosed embodiments.

### DETAILED DESCRIPTION

Reference will now be made in detail to specific embodiments of the present invention. Examples of these embodiments are illustrated in the accompanying drawings. While the embodiments will be described in conjunction with the drawings, it will be understood that the following description is not intended to limit the present invention to any one embodiment. On the contrary, the following description is intended to cover alternatives, modifications, and equivalents as may be included within the spirit and scope of the appended claims. Numerous specific details are set forth in order to provide a thorough understanding of the present invention.

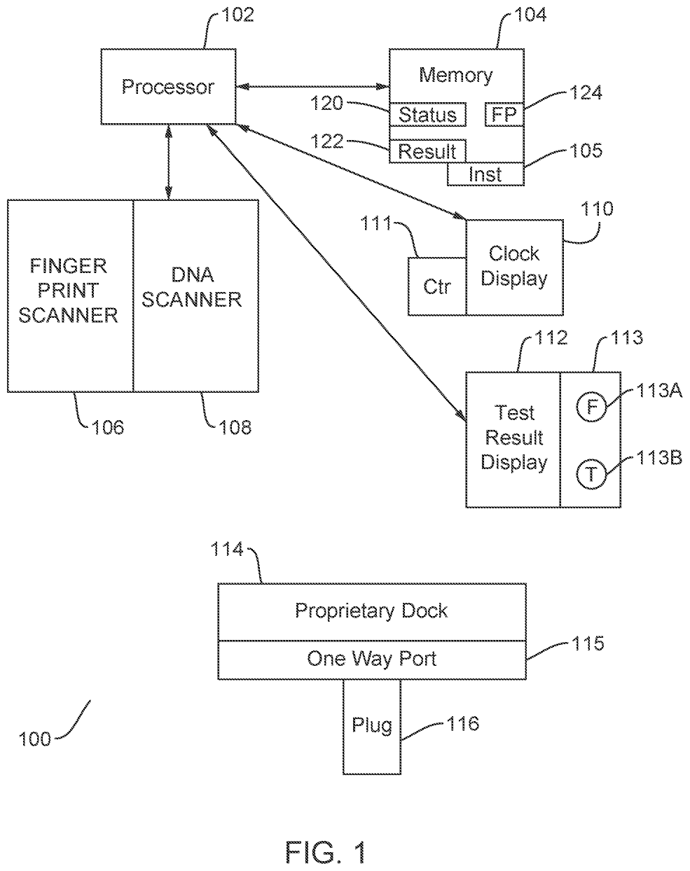

FIG. 1 illustrates a block diagram of the components of personal medical status device 100 according to the disclosed embodiments. FIG. 1 shows the preferred structural components of device 100 and preferred functional relationships between the components. The physical layout of device 100, however, may differ from what is shown in FIG. 1. Device 100 may include other features and components not disclosed by FIG. 1. For example, the user or patient may be tested to see if he/she has a virus condition. Perhaps the user has the virus but does not show outwards symptoms. The user may be asked to verify his/her medical status using device 100. The device also may require the user to have a medical test performed by a kit every so often, such as daily, to maintain the proper status. A user that fails to perform the tests may be indicated by the device as not certified such that the user may be made to have a test performed before proceeding.

Device 100 includes processor 102 and memory 104. Memory 104 stores instructions 105 that are executed by processor 102 to enable device 100 to determine, certify, and store test results from medical testing kits as well as authenticate a user of the device and his/her condition to a third party. Processor 102 also may execute instructions to enable the components of device 100 disclosed below. Memory 104 also may store relevant data to verify the medical status of the user.

Device 100 includes fingerprint scanner 106 that scans a fingerprint of the user to authenticate the user with a fingerprint file 124 stored in memory 104. Fingerprint scanner 106 validates that the user is the owner/verified user of device 100 and the attached test results, which may be stored as test results 122 in memory 104. Fingerprint scanner 106 may scan the fingerprint of the user. With the help of processor 102, fingerprint scanner 106 may compare the scanned fingerprint to a saved image file of the user's fingerprint stored as fingerprint file 124.

Fingerprint scanner 106 may execute software to provide functionality within device 100. The software may provide biometrics processing and analysis. Fingerprint scanner 106 may execute a fingerprint image quality algorithm that analyses a fingerprint image captured by the fingerprint scanner and assigns a quality value of 1 (highest quality) to 5 (lowest quality) to the image. Higher quality images produce significantly better performance with matching algorithms. Weights may be used with the fingerprint image quality algorithm as well.

A neural-network-based fingerprint pattern classification system may be implemented that automatically categorizes a fingerprint image captured by fingerprint scanner 106 into the class of arch, left or right loop, scar, tented arch, or whorl. The system includes the use of a robust multi-layered perceptron neural network. A minutiae detector may automatically locate and record ridge ending and bifurcations in a fingerprint image. The system include minutiae quality assessment based on local image conditions.

A large collection of image utilities may be implemented by fingerprint scanner 106 to support the processing of fingerprint images. Source code may be implemented for baseline JPEG, lossless JPEG, and the wavelet scalar quantization encoders and decoders. Utilities also are provided that support color component interleaving, colorspace conversion, and format conversion of legacy files in fingerprint databases.

Fingerprint scanner 106 may implement various algorithms. In some embodiments, fingerprint scanner 106 may implement a fingerprint matching algorithm that is a minutiae-based fingerprint matching algorithm. It will do both one-to-one and one-to-many matching operations. Alternatively, a fingerprint segmentation algorithm may implemented that will segment the four-finger plain impression found on the bottom of a fingerprint card into individual fingerprint images or it can be used to remove white space from a rolled fingerprint image.

Fingerprint scanner 106, in some embodiments, may implement a spectral validation/verification metric for fingerprint images that examines an input image with respect to expression of the level-1 ridge flow in a 1-D representation of its power spectrum. Relative power and frequency location of the dominant peak indicate the presence of a fingerprint in the image, the approximate sample rate, and provide rough measure of fingerprint quality.

Device 100 also may include DNA scanner 108 for two-factor authentication of the user. DNA information also may be stored on memory 104. Queries to device 100 would be confirmed with at least a fingerprint scan by scanner 106 and a DNA match performed by DNA scanner 108. Fingerprint scanner 106 may connect to processor 102 and act as the lock/unlock switch for device 100. Once authenticated by fingerprint scanner 106, device 100 may be queried for the certification state of a medical condition for the user.

Processor 102 and memory 104 may comprise the central system of device 100. Additional components may be included, but are not shown for brevity. The central system would be responsible for storing and displaying a certification status using test result display 112. Test result display 112 may provide a visual display of the status of the test results for the medical condition verified by device 100. Processor 102 may indicate to test result display 112 what status to display based on status 120 for test result 122 stored in memory 104.

Test result display 112 may include visual indicators 113, which may be display buttons or LEDs that emit a colored light related to the status. For example, visual indicators 113 may include a fingerprint indicator light 113A that turns “on” to display an authenticated, failed, incomplete, or out of date test result status. Test status visual indicator light 113B may turn “on” when the user is within a test window, close to an expired test status, or no results found.

Fingerprint indicator light 113A may display different colors to indicate the various statuses for the results from fingerprint scanner 106. A green light from light 113A indicates that the fingerprint of the user is authenticated. A yellow light from light 113A indicates that the fingerprint quality is low and the user should try again. A red light from light 113A indicates that fingerprint scanner 106 cannot authenticate the user or that the authentication failed. The user is not registered to use device 100.

Test result indicator light 113B illuminates once the fingerprint taken by fingerprint scanner is authenticated. Test result indicator light 113B provides a status of the test result stored on device 100. A green light from light 113B indicates that a clear test result is available with a period greater than 48 hours before expiration. A yellow light from light 113B indicates that a clear test result is available with a period less than 48 hours before expiration. A red light from light 113B indicates that no clear or unexpired rest result can be found.

Device 100 also provides information on the time elapsed since the user performed a test for the medical condition. The disclosed embodiments may prevent a user from providing a verified status by not testing regularly in order to maintain an acceptable test result status. A value may be stored in memory 104, for example, that indicates the period of time to elapse between tests. Clock display 110 may be placed outside device 100 to provide this information to the user as well as third parties.

A counter 111 may be connected to clock display 110 to count down the time until the next test. To reset counter 111, device 100 may need to receive a valid test result from a medical testing kit. Alternatively, the user may reset counter 111 manually but in conjunction with receipt of valid test results. Clock display 110 displays the time value determined by counter 111. For example, certification times for test results may be 12 or 24 hour loops that are defined on a periodic basis, such as one year. Thus, a user may need to have device 100 certified by a medical professional every year that test results are provided or verified by the device every 12 or 24 hours. Again, this process provides some level of validation that the user is following established medical protocols.

Device 100 also includes a proprietary dock 114. Other components in device 100 will not communicate with other systems except through proprietary dock 114. Proprietary dock 114 includes a one-way port 115 to receive test result 122, status 120, and other information that moves one-way into device 100. Device 100 may not send data to an attached medical testing kit or other system. Proprietary dock 114 also may include a plug 116 having a unique design to plug into a specified medical testing kit.

Thus, device 100 may be dedicated to providing results for a single or specified medical condition related to a specific set of medical testing kits. For example, an attached medical testing kit may indicate the presence of Covid-19 or other virus. Device 100 may be configured to provide results for various medical conditions. The user may specify a test type to authenticate. Device 100 may be configured to display which tests are being validated. Additional lights or a display may be included with test result display 112.

Referring to FIG. 1, proprietary dock 114 is responsible for providing certification status and timing to device 100. Proprietary dock 114 may implement a process to get a test status certification from an attached medical testing kit at device 100. Proprietary dock 114 also may implement a process to provide a method for a certified professional to stamp the certification with an anonymized credential. Proprietary dock 114 also may implement a process to set all the parameters of the certification for the medical condition. For example, a process may set the duration of the certification period, such as 24 hours or one year. Further, the type of certification may be set, such as which type of disease or condition is being tested.

Proprietary dock 114 also may be configured to create anonymized records of which certifications were issued to the user as well as the dates of the certifications. As information is provided to device 100, proprietary dock 114 may create a file or table that shows this information. The finalized set of information may be stored in memory 104. After an initial file is created by proprietary dock 114, it may be updated by the dock and its associated firmware while stored in memory 104. Further, proprietary dock 114 may need to operate with testing devices but could potentially avoid connections with those testing devices that rely on medical professional credentials. Thus, proprietary dock 114 may identify and validate whether to connect to a medical testing kit or device before any information is provided thereto.

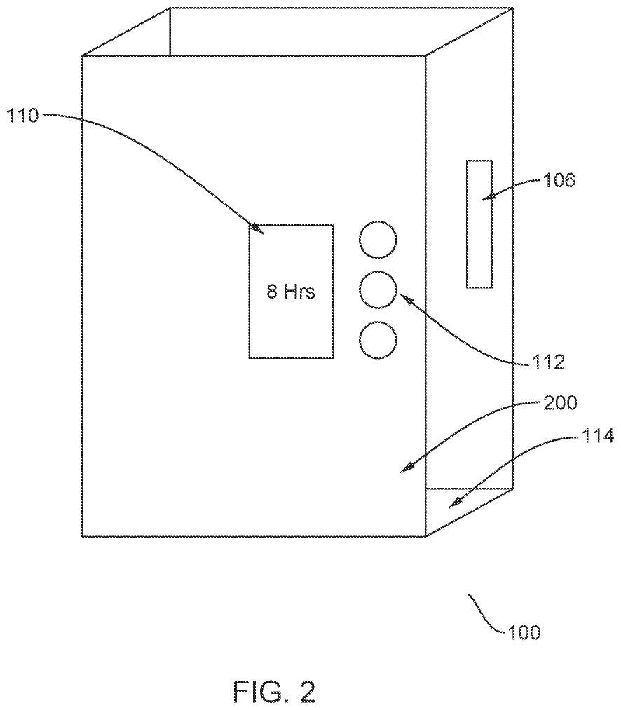

FIG. 2 depicts a personal medical status device 100 according to the disclosed embodiments. FIG. 2 may provide a perspective view of device 100. Device 100 includes the components disclosed by FIG. 1. Device 100 also includes a casing 200 to enclose the components as well as provide a structure to allow fingerprint scanner 106 to capture scans of fingerprints. Device 100 may include an aperture or opening to receive a finger such that fingerprint scanner 106 is aligned with the portion of the finger to capture its fingerprint.

FIG. 2 also depicts clock display 110 showing how many hours left until the next test result should be captured and provided to device 100. The visual indicators for test result display 112 provide the information on the status in a visual manner. Thus, the information regarding the medical condition may be provided quickly and without print outs, displayed confidential information, or the like. Proprietary dock 114 may be located at the bottom of device 100.

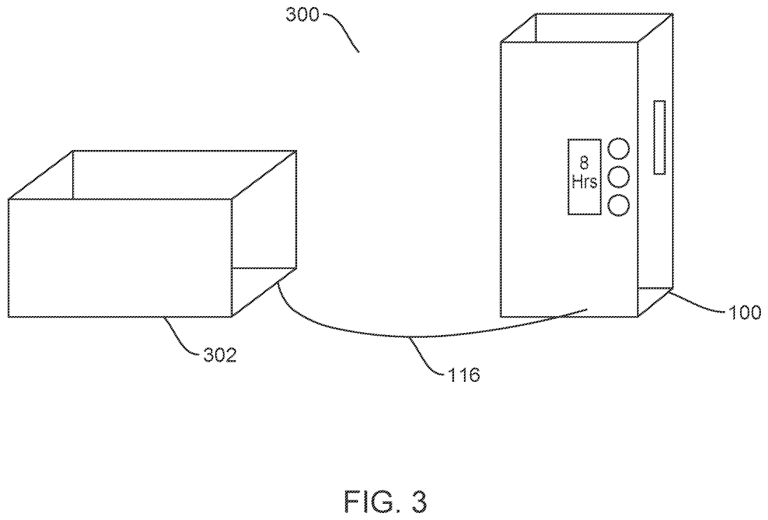

FIG. 3 depicts a medical testing and status system 300 according to the disclosed embodiments. System 300 includes medical testing kit 302 that works in conjunction with personal medical status device 100. Each home may have an FDA approved home testing kit 302. The user performs medical or health tests using the approved testing kit 302. The user may then plug device 100 into testing kit 302 to transfer the determined status 120 and time of the test. Device 100 may use plug 116 of proprietary dock 114 to connect to testing kit 302.

The user may verify the validity of the test result and status with a fingerprint match using fingerprint scanner 106. The disclosed embodiments also enable the use of DNA matching for the test results on device 100 using DNA scanner 108 for two-factor verification using fingerprint and DNA. Test result 122 and status 120 are received by processor 102, which sends instructions to test result display 112. The appropriate visual indicator 113 is lit to show the status of the user or patient. Further, clock display 110 shows the elapsed time since the last test or the remaining time until the next test.

Device 100 may be detached from testing kit 302 so that it is portable. Device 100 may be carried with the user and the results of the medical tests performed by testing kit 302 shared when asked. Fingerprint validation may be used to share these results using device 100. Thus, a user does not need to store sensitive medical information on a smart phone or other device running applications that may reveal this information. Further, device 100 may not be plugged into or connected with other devices aside from testing kit 302 to modify, remove, or obtain test results 122 or status 120. Moreover, the user may provide his/her status without revealing any personal information to a third party. The device merely shows the status using visual indicators 113. The user does not even need to provide a name or other personal information.

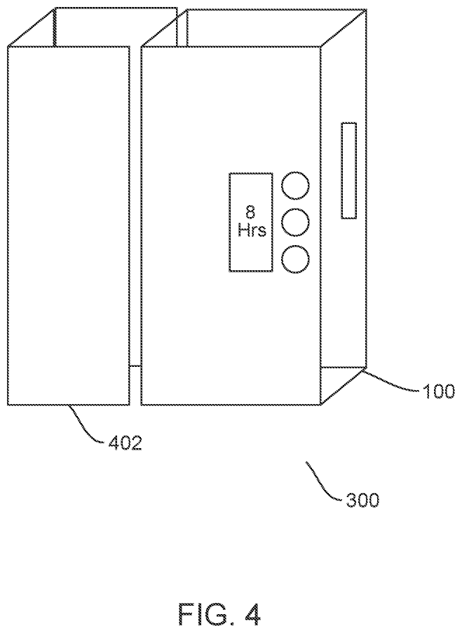

FIG. 4 depicts a personal medical status device 100 attached to a mini-testing kit 402 according to the disclosed embodiments. Medical testing and status system 300 includes mini-testing 402 instead of testing kit 302. The user tests their medical or health status with an attached miniature testing kit. The miniature testing kit 402 may allow the testing to occur outside the home or office. The user may take mini-testing kit 402 on trips or excursions without the need to connect to a medical testing kit 302 that is not portable. In some embodiments, mini-testing kit 402 also may connect to testing kit 302 for exchanging data and information, as well as synchronize test results.

For example, the user may depart on a business trip for several days. The user takes system 300 with him/her as mini-testing kit 402 and device 100. During the day, the user takes device 100 along to authenticate status and provide the needed information regarding the medical condition. In the evening, the user may perform the needed medical tests to generate the test results required by device 100 to maintain certification. The user may do this as much as needed during the trip. Upon return home, the user may connect mini-testing kit 402 with testing kit 302 to synchronize the generated test results.

The disclosed systems and device may be used in conjunction with hospital outpatient treatment. For example, in the event that a person is infected with a disease, especially a highly contagious disease such as Covid-19, the medical testing kit and the personal medical status device may be used to monitor treatment and verify the status of the patient. If the person shows the proper immune response and is no longer contagious, then the hospital may give the user a long term acceptable, or “green,” certification based on their immune system. Device 100 then would how the “green” status using the appropriate visual indicator 113 and clock display 110 with counter 111 would reset automatically every 12 hours to protect privacy.

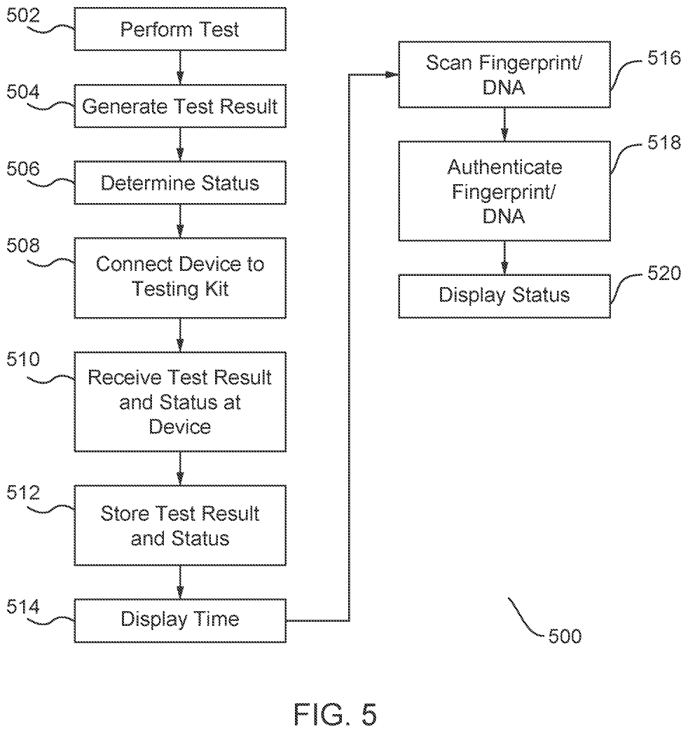

FIG. 5 depicts a flowchart 500 for using the medical testing and status system according to the disclosed embodiments. For illustrative purposes, medical testing and status system 300 using device 100 and medical testing kit 302 may be referred in the following discussion.

Step 502 executes by performing a medical or health test using medical testing kit 302. The test relates to a condition of the user of the testing kit, such as whether he/she is infected with a disease. Step 504 executes by generating a test result based on the test. The test result may include data related to the testing done. Step 506 executes by determining a status for the condition based on the test result. For example, the status may be passed, failed, or inconclusive. Other statuses may include infected, clean, or results not available.

Step 508 executes by connecting personal medical status device 100 to medical testing kit 302. A plug 116 from proprietary dock 114 may be plugged into testing kit 302. Step 510 executes by receiving the test result and the status for the performed test. In some embodiments, only the status is provided as the test results may not be needed by device 100. Such information may not want to be stored on device 100. Step 512 executes by storing the test result and the status on device 100 as test result 122 and status 120. The disclosed embodiments also may certify the received information from medical testing kit 302.

Step 514 executes by displaying time from the previous test or time left until the next test in clock display 110. Device 100 provides this information to alert the user or a third party that another test should be done. If counting from the time of the previous test, then the user or the third party may see the time and make a determination whether the result is still valid.

When the user wishes to authenticate his/her status, then step 516 executes by scanning the fingerprint using fingerprint scanner 106. In some embodiments, DNA scanner 108 may obtain DNA information from the user. Step 518 executes by authenticating the user by matching the captured fingerprint information to the fingerprint file 124 stored on device 100 for the user. The same action may be taken with the DNA information. If the user is authenticated, then step 520 executes by displaying the status of the user via test result display 112. In some embodiments, steps 516-20 may be executed when connecting to medical testing kit 302 and receiving test results and statuses for medical tests.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A personal medical status device comprising:
a processor and a memory connected to the processor, the memory storing instructions executable by the processor to receive and process a test result received from a medical testing kit, wherein the memory stores the test result provided by the medical testing kit and a status associated with the test result;
a fingerprint scanner to scan a fingerprint of a user and to authenticate the user with a fingerprint file stored in the memory;
a clock display to indicate a time related to the test result provided by the medical testing kit;
a test result visual display to indicate the status of the test result for the user; and
a proprietary dock to attach the medical testing kit, wherein the proprietary dock receives the test result from the medical testing kit and certifies the test result, the proprietary dock including a tamper proof feature and only allows data to transfer in one direction from the medical testing kit.

2. The personal medical status device of claim 1, further comprising a DNA verification component to verify the DNA of the user.

3. The personal medical status device of claim 1, wherein the clock displays an elapsed time as the time related to the test result.

4. The personal medical status device of claim 1, wherein the personal medical status device is a handheld device.

5. The personal medical status device of claim 1, wherein the medical testing kit is attached to the personal medical status device.

6. A medical testing and status system comprising:
a medical testing kit to determine a status for a medical condition of a user; and
the personal medical status device of claim 1 connected to the medical testing kit.

7. The medical testing and status system of claim 6, wherein the medical testing device is connected to the personal medical status device by the proprietary dock.

8. The medical testing and status system of claim 6, wherein the medical testing device attaches to a side of the personal medical status device.

9. A method comprising:
performing a medical test for a condition of a patient using a medical testing kit;
connecting a personal medical status device to the medical testing kit;
receiving a test result for the medical test at the personal medical status device using a proprietary dock, wherein the test result is certified;
storing the test result on the personal medical status device;
displaying a time based on the receipt of the test result with a clock on the personal medical status device;
indicating a status of the test result using a test result visual display on the personal medical status device;
authenticating the status of the patient using a fingerprint scanner on the personal medical status device, wherein the status is displayed after verification by the fingerprint scanner.

10. The method of claim 9, further comprising voiding the test result if the personal medical status device is opened.

11. The method of claim 9, further comprising capturing an image of a fingerprint by the fingerprint scanner.

12. The method of claim 9, wherein authenticating the status includes implementing an algorithm to match the captured image with a fingerprint file.

13. The method of claim 12, wherein the fingerprint file is stored in a memory on device 100.
